(19) United States
(12) Patent Application Publication
(10) Pub. No.: US 2025/0216330 A1
(43) Pub. Date: Jul. 3, 2025

(54) FLUORESCENCE INTENSITY MEASUREMENT DEVICE, AND METHOD OF MEASURING FLUORESCENCE INTENSITY

(71) Applicant: Tianma Japan, Ltd., Kawasaki (JP)

(72) Inventors: Kazunori MASUMURA, Kawasaki (JP); Ayuko IMAI, Kawasaki (JP); Ken SUMIYOSHI, Kawasaki (JP)

(73) Assignee: Tianma Japan, Ltd., Kawasaki (JP)

(21) Appl. No.: 18/964,807

(22) Filed: Dec. 2, 2024

(30) Foreign Application Priority Data
Dec. 27, 2023 (JP) 2023-220652

(51) Int. Cl. G01N 21/64

(52) U.S. Cl. CPC G01N 21/6428; G01N 2021/6439; G01N 2201/12

(57) ABSTRACT

A fluorescence intensity measurement device includes: a light source to emit, to a measurement target, a plurality of pieces of pulse-shaped excitation light of which a pulse width is modulated; a CMOS image sensor to detect, as an image for each pulse of a piece of the plurality of pieces of pulse-shaped excitation light, fluorescence emitted from the measurement target by the piece of the plurality of pieces of pulse-shaped excitation light; and a controller to calculate a fluorescence intensity of the fluorescence. The controller calculates the fluorescence intensity of the fluorescence, based on two duty ratios of the two pieces of pulse-shaped excitation light having different pulse widths, and two light intensities obtained from the images detected by the two pieces of pulse-shaped excitation light.

### CROSS-REFERENCE TO RELATED APPLICATIONS

This application claims the benefit of Japanese Patent Application No. 2023-220652, filed on Dec. 27, 2023, the entire disclosure of which is incorporated by reference herein.

### FIELD OF THE INVENTION

The present disclosure relates generally to a fluorescence intensity measurement device and a method of measuring a fluorescence intensity.

### BACKGROUND OF THE INVENTION

In this related art, an analysis method, an observation method, and the like using fluorescence are known. For example, a fluorescence polarization immunoassay (FPIA) method that uses antigen-antibody reactions to detect a detection substance is known. Unexamined Japanese Patent Application Publication No. H03-103765 describes a method for calculating the concentration of a measurement antigen (detection substance) from the measured degree of polarization of fluorescence.

Further, in this related art, a fluorescence observation device that irradiates a measurement target with excitation light, and captures an image by fluorescence emitted from the measurement target is known. For example, Unexamined Japanese Patent Application Publication No. 2000-210246 describes a fluorescence observation device that includes excitation light emitting means for emitting excitation light and fluorescence image capturing means for detecting fluorescence to capture a fluorescence image of a living body.

The fluorescence observation device of Unexamined Japanese Patent Application Publication No. 2000-210246 further includes modulation means for modulating intensity of the fluorescence entered capturing means based on a predetermined periodic function, and analysis means for obtaining, from time-series image data output by the fluorescence image capturing means, a characteristic value corresponding to an amplitude of periodic change in fluorescence intensity per each pixel. In the fluorescence observation device of Unexamined Japanese Patent Application Publication No. 2000-210246, a fluorescence image having a high signal-to-noise ratio (high SN ratio) is obtained by performing intensity modulation of the excitation light to a sine wave form, performing Fourier transform of the time-series image data per each pixel, and then removing an influence, on the characteristic value, due to an external disturbance such as background light, noise, or the like.

If a CMOS image sensor is used in detection of fluorescence, the sensitivity can be increased by, for example, cooling the CMOS image sensor and lengthening an exposure time of the CMOS image sensor. However, there are limits in cooling of the CMOS image sensor. Further, if the exposure time is lengthened, noise (also referred as to amplifier light, amplifier grow, etc.) by heat, infrared light, or the like from a peripheral circuit (amplifier circuit, AD converter, etc.) of the CMOS image sensor notably appears. Noise caused by the peripheral circuit can be removed by measurement of background, but the background should be measured at each measurement for precise measurement of fluorescence.

Further, as in Unexamined Japanese Patent Application Publication No. 2000-210246, the intensity of excitation light should accurately be modulated if the intensity modulation of excitation light and Fourier transform are used to remove noise. For example, if the intensity-modulated excitation light includes a direct current component, it is difficult to distinguish a fluorescence component from noise caused by peripheral circuit, background light, or the like. In a case where a light source of excitation light includes light emitting diodes (LEDs), accurate modulation of the intensity of excitation light is difficult since the voltage-current characteristics of the LEDs are in a non-linear manner.

### SUMMARY OF THE INVENTION

A fluorescence intensity measurement device according to a first aspect of the present disclosure includes:
a light source to emit, to a measurement target, a plurality of pieces of pulse-shaped excitation light of which a pulse width is modulated;
a CMOS image sensor to detect, as an image for each pulse of a piece of the plurality of pieces of pulse-shaped excitation light, fluorescence emitted from the measurement target by the piece of the plurality of pieces of pulse-shaped excitation light; and
a controller to calculate a fluorescence intensity of the fluorescence, wherein
the controller calculates the fluorescence intensity of the fluorescence, based on two duty ratios of two pieces of pulse-shaped excitation light having different pulse widths of the plurality of pieces of pulse-shaped excitation light, and two light intensities obtained from the images detected by the two pieces of pulse-shaped excitation light.

A method of measuring a fluorescence intensity according to a second aspect of the present disclosure includes:
emitting, to a measurement target, a plurality of pieces of pulse-shaped excitation light of which a pulse width is modulated;
detecting, by the CMOS image sensor as an image for each pulse of a piece of the plurality of pieces of pulse-shaped excitation light, fluorescence emitted from the measurement target by the piece of the plurality of pieces of pulse-shaped excitation light; and
calculating a fluorescence intensity of the fluorescence, wherein
in the calculating,
the fluorescence intensity of the fluorescence is calculated based on two duty ratios of two pieces of pulse-shaped excitation light having different pulse widths of the plurality of pieces of pulse-shaped excitation light, and two light intensities obtained from the images detected by the two pieces of pulse-shaped excitation light.

It is to be understood that both the foregoing general description and the following detailed description are exemplary and explanatory and are not restrictive of this disclosure.

### BRIEF DESCRIPTION OF DRAWINGS

A more complete understanding of this application can be obtained when the following detailed description is considered in conjunction with the following drawings, in which:

FIG. 1 is a schematic diagram illustrating a fluorescence intensity measurement device according to Embodiment 1;

FIG. 2 is a plan view illustrating a microdevice according to Embodiment 1;

FIG. 3 is a cross-sectional view of the microdevice illustrated in FIG. 2 taken along a line C-C;

FIG. 4 is a block diagram illustrating a configuration of a controller according to Embodiment 1;

FIG. 5 is a schematic view illustrating excitation light according to Embodiment 1;

FIG. 6 is a diagram illustrating a light intensity according to Embodiment 1;

FIG. 7 is another diagram illustrating a light intensity according to Embodiment 1;

FIG. 8 is a diagram illustrating a fluorescence intensity according to Embodiment 1;

FIG. 9 is a diagram illustrating a hardware configuration of the controller according to Embodiment 1;

FIG. 10 is a flowchart illustrating detection processing according to Embodiment 1;

FIG. 11 is a flowchart illustrating fluorescence intensity measurement processing according to Embodiment 1;

FIG. 12 is a schematic diagram illustrating an example of excitation light according to Embodiment 2;

FIG. 13 is a diagram illustrating a light intensity according to Embodiment 2; and

FIG. 14 is a diagram illustrating a fluorescence intensity according to Embodiment 2.

### DETAILED DESCRIPTION OF THE INVENTION

Hereinafter, a fluorescence intensity measurement device according to various embodiments is described with reference to the drawings.

### Embodiment 1

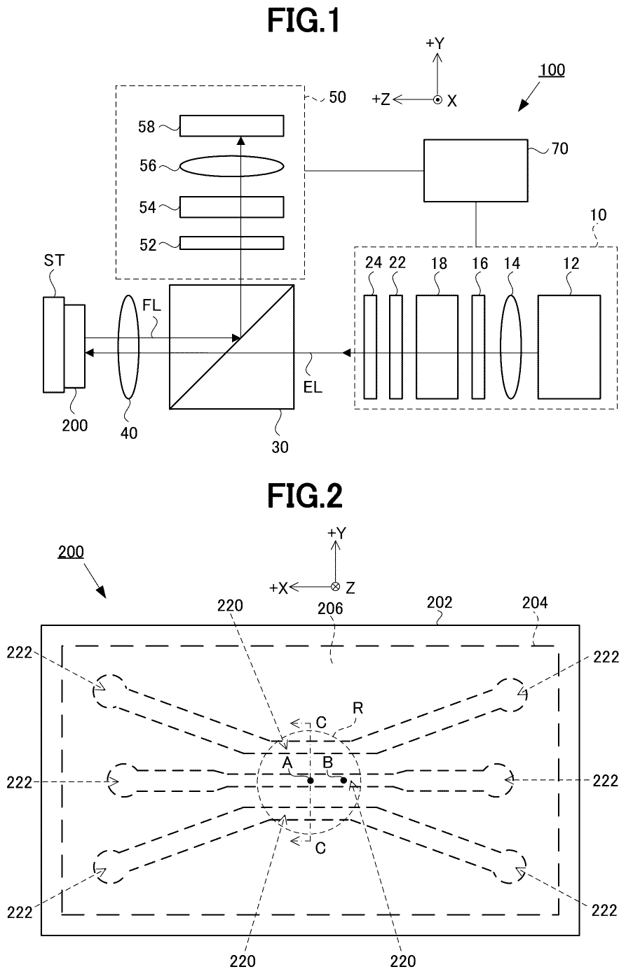

Firstly, a fluorescence intensity measurement device 100 according to the present embodiment is described with reference to FIGS. 1 to 11. In one example, the fluorescence intensity measurement device 100 is used in detection, using a fluorescence polarization immunoassay method, of a detection substance contained in measurement target solution to be measured. The measurement target solution corresponds to a measurement target to be measured.

As illustrated in FIG. 1, the fluorescence intensity measurement device 100 includes a light source unit 10, a dichroic mirror 30, an objective lens 40, a detector 50, and a controller 70. The light source unit 10 emits excitation light EL of which a pulse width is modulated and that is linearly polarized. The excitation light EL is a plurality of pieces of pulse-shaped excitation light. The excitation light EL emitted from the light source unit 10 irradiates, through the dichroic mirror 30 and the objective lens 40, the measurement target solution that is introduced to microchannels 220 of a microdevice 200, described later. The detector 50 detects, as an image, from among pieces of fluorescence FL emitted from the measurement target solution, a piece of fluorescence FL having a polarization direction in a predetermined direction. The controller 70 controls the various components of the fluorescence intensity measurement device 100. Further, the controller 70 calculates, based on a duty ratio of the excitation light EL and the light intensity obtained from the detected image, a fluorescence intensity of the piece of fluorescence FL having the polarization direction in the predetermined direction. The controller 70 further calculates a concentration of the detection substance by calculating a degree of polarization P of the measurement target solution.

Note that, in the present disclosure, to facilitate comprehension, in the fluorescence intensity measurement device 100 of FIG. 1, the left direction (the left direction on paper) is referred to as the “+Z direction”, the up direction (the up direction on paper) is referred to as the “+Y direction”, and the direction perpendicular to the +Y direction and the +Z direction (the front direction on paper) is referred to as the “+X direction.” The excitation light EL is light that excites a fluorescence-labeled derivative, described later, and that causes the fluorescence-labeled derivative to emit fluorescence. The light source unit 10, the dichroic mirror 30, and the objective lens 40 form a lighting optical system, and the objective lens 40, the dichroic mirror 30, and the detector 50 form an observation optical system.

Firstly, the measurement target solution and the microdevice 200 are described. The measurement target solution includes a detection substance, a fluorescence-labeled derivative, and an antibody. The detection substance is the detection target of the fluorescence intensity measurement device 100. The detection substance may be any compound detectable in a fluorescence immunoassay. Examples of the detection substance include antibiotics, bioactive substances, mycotoxins, and the like. Specific examples of the detection substance include β-lactoglobulin, chloramphenicol, deoxynivalenol, and the like. The fluorescence-labeled derivative is a derivative obtained by fluorescently labeling the detection substance with a fluorescent substance. The fluorescence-labeled derivative can be obtained by using a known method to bind the fluorescent substance to the detection substance. The fluorescent substance is, for example, fluorescein (wavelength of excitation light EL: 494 nm, wavelength of fluorescence FL: 521 nm) or the like. The antibody binds specifically to the detection substance due to an antigen-antibody reaction. In one example, the antibody is obtained by inoculating a host animal (for example, a mouse or a cow) with the detection substance, and then, collecting the antibodies in the blood produced by the host animal and purifying the collected antibodies in the blood. Alternatively, a commercially available antibody can be used as the antibody.

The detection substance and the fluorescence-labeled derivative bind specifically to the antibody due to the antigen-antibody reaction in a competitive manner. In fluorescence polarization immunoassays, the degree of polarization P of the fluorescence FL emitted by the fluorescence-labeled derivative included in the measurement target solution is obtained. The concentration of the detection substance can be calculated from the obtained degree of polarization P and a calibration curve that is created in advance.

The fluorescence-labeled derivative not bound to the antibody moves vigorously in the measurement target solution and, as such, when the fluorescence-labeled derivative not bound to the antibody is irradiated with the excitation light EL that is polarized light, fluorescence FL is randomly emitted. Meanwhile, movement in the measurement target solution of the fluorescence-labeled derivative bound to the antibody is limited and, as such, when the fluorescence-labeled derivative bound to the antibody is irradiated with the excitation light EL that is polarized light, fluorescence FL biased in the polarization direction of the excitation light EL is emitted. A fluorescence intensity Ih of the fluorescence FL having a polarization direction parallel to the polarization direction of the excitation light EL and a fluorescence intensity Iv of the fluorescence FL having a polarization direction perpendicular to the polarization direction of the excitation light EL are measured, and the bias of the fluorescence intensity is calculated as the degree of polarization P. The degree of polarization P is dependent on the amount of the fluorescence-labeled derivative bound to the antibody and, as such, the concentration of the detection substance can be calculated from the obtained degree of polarization P and the calibration curve that is created in advance. Note that the degree of polarization P is expressed as

P=(Ih−Iv)/(Ih+Iv).

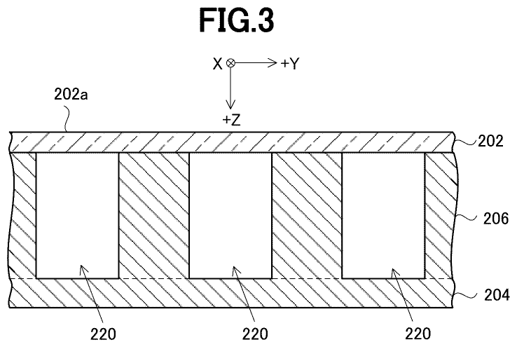

As illustrated in FIGS. 2 and 3, the microdevice 200 includes a first substrate 202, a second substrate 204, a partition wall 206, and three microchannels 220. The measurement target solution is introduced into each of the microchannels 220. The microdevice 200 is placed on a stage ST of the fluorescence intensity measurement device 100.

The first substrate 202 of the microdevice 200 is implemented as a flat plate-like quartz glass substrate. The excitation light EL emitted from the light source unit 10 of the fluorescence intensity measurement device 100 enters the microdevice 200 from the first substrate 202. The excitation light EL falls on a measurement region R illustrated in FIG. 2 from the −Z direction, and perpendicularly enters a main surface 202a of the first substrate 202.

The second substrate 204 of the microdevice 200 is implemented as a flat plate-like substrate. The second substrate 204 is formed from a material that has low autofluorescence. In one example, the second substrate 204 is formed from carbon black-containing polydimethylsiloxane (PDMS). The second substrate 204 faces the first substrate 202. The second substrate 204 and the first substrate 202 sandwich the partition wall 206.

The partition wall 206 of the microdevice 200 is sandwiched by the first substrate 202 and the second substrate 204 to form the microchannels 220. The partition wall 206 is formed from a material that has low autofluorescence. Additionally, it is preferable that the partition wall 206 is formed from a material that absorbs light such as the excitation light EL, the fluorescence FL, and the like. In the present embodiment, the partition wall 206 is formed integrally with the second substrate 204.

The microchannels 220 of the microdevice 200 extend parallel to the X direction in the measurement region R. In one example, a width of each of the microchannels 220 in the measurement region R is 200 μm. Each of the microchannels 220 includes two openings 222 that penetrate the second substrate 204 and the partition wall 206. The measurement target solution is introduced or discharged through the openings 222.

Next, the various components of the fluorescence intensity measurement device 100 are described. As illustrated in FIG. 1, the light source unit 10 of the fluorescence intensity measurement device 100 includes a light source 12, a condenser lens 14, an iris 16, a collimator 18, a polarization filter 22, and an excitation light filter 24.

The light source 12 emits, in the +Z direction, light including the excitation light EL, that is, the excitation light EL. In one example, the light source 12 includes an LED element. The light source 12 is controlled by the controller 70, and emits pulse-shaped light including pulse-shaped excitation light EL of which the pulse width is modulated based on a pulse width modulation (PWM) signal from the controller 70. The excitation light EL of which the pulse width is modulated is described later.

Light emitted from the light source 12 is focused by the condenser lens 14 and then, passes through the iris 16. The iris 16 reduces the influence of external light. The external light is light other than the light emitted from the light source 12. The light that has passed through the iris 16 enters the collimator 18.

The collimator 18 converts the entering light to parallel light. The light that is converted to parallel light enters the polarization filter 22.

The polarization filter 22 emits, from among the entering light, light having a polarization direction in a predetermined direction. The light that is emitted from the polarization filter 22 enters the excitation light filter 24. In the present embodiment, the polarization filter 22 emits light having a polarization direction in the X direction. Specifically, the light, including the excitation light EL, that enters the polarization filter 22 is emitted as linear polarized light having the polarization direction in the X direction, and enters the excitation light filter 24. In one example, the polarization filter 22 is implemented as a polarizing plate.

The excitation light filter 24 removes, from the light emitted from the light source 12, light other than the excitation light EL. In one example, the excitation light filter 24 is implemented as a bandpass filter.

Accordingly, the plurality of pieces of pulse-shaped excitation light EL of which the pulse width is modulated and that has the polarization direction in the X direction is emitted from the light source unit 10 in the +Z direction. The pulse-shaped excitation light EL of which the pulse width is modulated and that has the polarization direction in the X direction enters the dichroic mirror 30.

The dichroic mirror 30 of the fluorescence intensity measurement device 100 transmits, in the +Z direction, the pulse-shaped excitation light EL of which the pulse width is modulated and that has the polarization direction in the X direction and reflects, to the detector 50 (+Y direction), the fluorescence FL emitted from the microdevice 200. The objective lens 40 of the fluorescence intensity measurement device 100 focuses the fluorescence FL and the excitation light EL transmitted through the dichroic mirror 30.

The pulse-shaped excitation light EL of which the pulse width is modulated and that has the polarization direction in the X direction is emitted on the measurement region R of the microdevice 200 through the dichroic mirror 30 and the objective lens 40. As a result, the fluorescence FL is emitted from the measurement target solution (the fluorescence-labeled derivative) introduced into the microchannels 220 of the microdevice 200. The fluorescence FL travels in the +Y direction through the objective lens 40 and the dichroic mirror 30, and enters the detector 50 (an absorption filter 52, described later).

The detector 50 of the fluorescence intensity measurement device 100 is disposed on the +Y side of the dichroic mirror 30. The detector 50 includes the absorption filter 52, a polarization adjustment element 54, an imaging lens 56, and a CMOS image sensor 58.

The absorption filter 52 separates the fluorescence FL emitted from the microdevice 200 from light such as scattered light, leaked light, and the like, and transmits the fluorescence FL. In one example, the absorption filter 52 is implemented as a bandpass filter. The fluorescence FL emitted from the absorption filter 52 enters the polarization adjustment element 54.

The polarization adjustment element 54 adjusts the fluorescence FL transmitted through the absorption filter 52 as linearly polarized light. Further, the polarization adjustment element 54 switches the polarization direction of the fluorescence FL that is linearly polarized and enters the CMOS image sensor 58 to a direction parallel to the polarization direction (X direction) of the excitation light EL emitted from the light source unit 10 and a direction (Z direction) perpendicular to the polarization direction of the excitation light EL emitted from the light source unit 10. The fluorescence FL that is linearly polarized enters the CMOS image sensor 58 through the imaging lens 56. In one example, the polarization adjustment element 54 is implemented as a twisted nematic (TN) liquid crystal element.

The CMOS image sensor 58 detects, based on a trigger signal from the controller 70, for each pulse of a piece of the plurality of pieces of pulse-shaped excitation light EL, a spatial distribution of fluorescence intensity of the fluorescence FL having the predetermined polarization direction (the polarization direction in the X direction or the polarization direction in the Z direction), as an image. That is, the CMOS image sensor 58 detects, with synchronization with emitting of the excitation light EL, the spatial distribution of the fluorescence FL having the predetermined polarization direction as the image. The CMOS image sensor 58 generates image data representing an image captured, to transmit the image data to the controller 70. Note that the main surface 202a of the first substrate 202 of the microdevice 200 and an imaging surface of the CMOS image sensor 58 have an image-forming relationship.

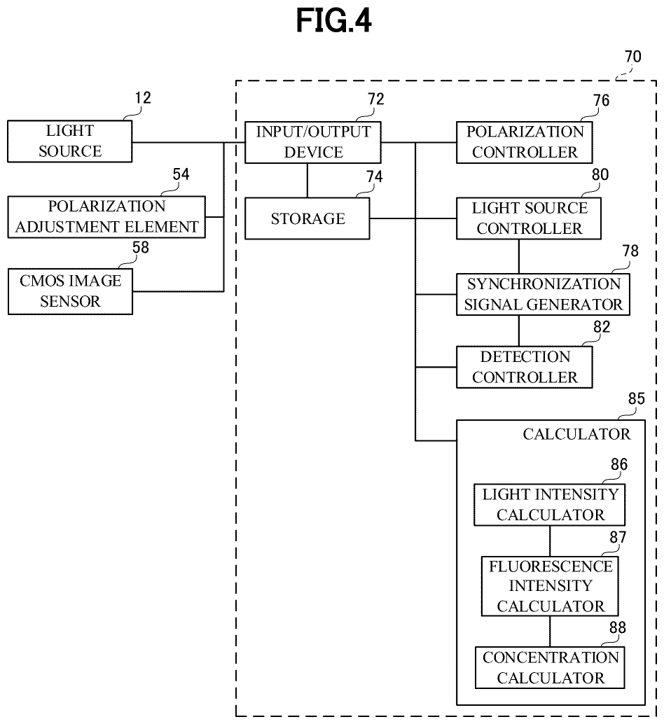

The controller 70 of the fluorescence intensity measurement device 100 controls the various components of the fluorescence intensity measurement device 100. Further, the controller 70 calculates, based on a duty ratio of the pulse-shaped excitation light EL and the light intensity, a fluorescence intensity of the fluorescence FL having the predetermined polarization direction, that is, the predetermined polarization direction in the X direction or the polarization direction in the Z direction. The light intensity is obtained from the image detected by the CMOS image sensor 58, that is, the image date transmitted from the CMOS image sensor 58. Further, the controller 70 calculates the degree of polarization P from the calculated fluorescence intensity of the fluorescence FL, and calculates the concentration of the detection substance from the degree of polarization P. As illustrated in FIG. 4, the controller 70 includes an input/output device 72, a storage 74, a polarization controller 76, a synchronization signal generator 78, a light source controller 80, a detection controller 82, and a calculator 85. The calculator 85 includes a light intensity calculator 86, a fluorescence intensity calculator 87, and a concentration calculator 88.

The input/output device 72 inputs/outputs signals, data, and the like between the controller 70 and the various components.

The storage 74 stores programs, image data transmitted from the CMOS image sensor 58, data expressing a calibration curve of the degree of polarization P and the concentration of detection substance, and the like.

The polarization controller 76 controls the polarization adjustment element 54 of the fluorescence intensity measurement device 100, thereby controlling the polarization direction of the fluorescence FL incident on the CMOS image sensor 58. For example, the polarization controller 76 first adjusts the polarization direction of the fluorescence FL entering the CMOS image sensor 58 as the X direction. After the CMOS image sensor 58 detecting the fluorescence intensity of the fluorescence FL having the polarization direction in the X direction as the image, the polarization controller 76 switches the polarization direction of the fluorescence FL entering the CMOS image sensor 58 to the Z direction.

The synchronization signal generator 78 generates a synchronization signal. The synchronization signal causes synchronization between the emitting (irradiation) of the excitation light EL from the light source 12 (light source unit 10) and the detection (imaging) by the CMOS image sensor 58. The synchronization signal generator 78 transmits the synchronization signal to the light source controller 80 and the detection controller 82.

The light source controller 80 transmits the PWM signal to the light source 12 based on the synchronization signal transmitted from the synchronization signal generator 78. The light source 12 emits the pulse-shaped light including the pulse-shaped excitation light EL of which the pulse width is modulated based on the PWM signal. The light, of which the pulse width is modulated, emitted from the light source 12 is emitted, through the polarization filter 22, the excitation light filter 24, and the like, on the measurement target solution (microdevice 200) as the pulse-shaped excitation light EL of which the pulse width is modulated and that has the polarization direction in the X direction.

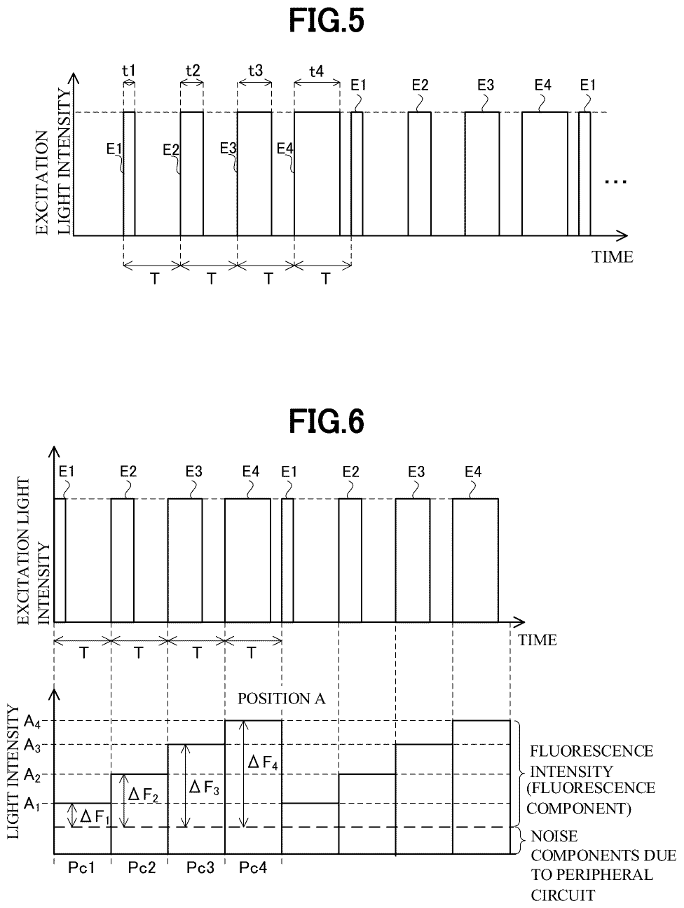

In the present embodiment, the pieces of pulse-shaped excitation light E1 to E4 of which the pulse widths t1 to t4 are sequentially broaden are sequentially emitted on the measurement target solution at the interval T. The pieces of pulse-shaped excitation light E1 to E4 are illustrated in FIG. 5. Then, the pulse train of the pieces of excitation light E1 to E4 is repeatedly emitted on the measurement target solution. Here, the pieces of excitation light E1 to E4 each have a polarization direction in the X direction, and have an equal excitation light intensity.

The detection controller 82 transmits the trigger signal to the CMOS image sensor 58 based on the synchronization signal transmitted from the synchronization signal generator 78. The CMOS image sensor 58 detects, based on the trigger signal, each of the pieces of fluorescence FL at the interval T as an image. The pieces of fluorescence FL each have the predetermined polarization direction (the polarization direction in the X direction or the polarization direction in the Z direction) emitted from the measurement target solution by the corresponding piece of excitation light E1 to E4. In the following, the light intensity calculator 86 and the fluorescence intensity calculator 87 are described using an example of the fluorescence FL having the polarization direction in the X direction. Here, an image detected by the excitation light E1 is defined as Pc1, an image detected by the excitation light E2 is defined as Pc2, an image detected by the excitation light E3 is defined as Pc3, and an image detected by the excitation light E4 is defined as Pc4.

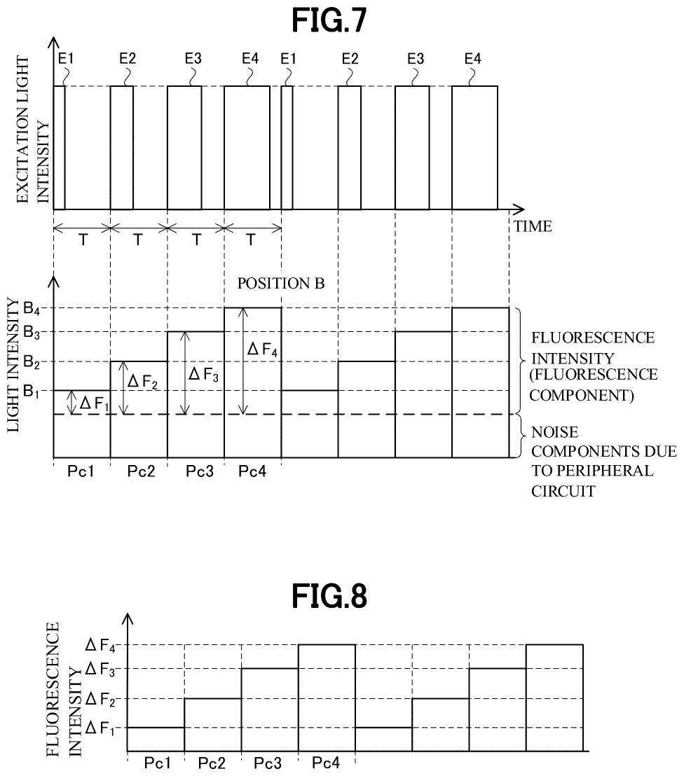

The light intensity calculator 86 of the calculator 85 calculates light intensity (brightness) of the microchannels 220 at the predetermined position for each of the images Pc1 to Pc4 detected by the CMOS image sensor 58. For example, at position A of the microchannel 220 illustrated in FIG. 2, the light intensities Al to A4 illustrated in FIG. 6 are calculated. Further, at position B of the microchannel 220 illustrated in FIG. 2, the light intensities B1 to B4 illustrated in FIG. 7 are calculated. As illustrated in FIGS. 6 and 7, the light intensities calculated from the images Pc1 to Pc4 include the fluorescence intensities ΔF1 to ΔF4 of the fluorescence FL having the polarization direction in the X direction and noise components. The fluorescence intensities ΔF1 to ΔF4 are fluorescence components in accordance with the pulse widths t1 to t4 of the pieces of excitation light E1 to E4. The noise components are due to heat, infrared light, and the like from a peripheral circuit of the CMOS image sensor 58, and are hereinafter referred to as noise components due to the peripheral circuit. The fluorescence intensities ΔF1 to ΔF4 of the fluorescence FL are independent of the position of the microchannels 220, but the magnitude of the noise components due to the peripheral circuit is dependent on the position of the microchannels 220.

The fluorescence intensity calculator 87 of the calculator 85 calculates, from the duty ratios of the pieces of excitation light E1 to E4 and the light intensities Al to A4 and B1 to B4 calculated from the images Pc1 to Pc4 by the light intensity calculator 86, the fluorescence intensities ΔF1 to ΔF4 of the fluorescence FL having the polarization direction in the X direction emitted from the measurement target solution by the respective pieces of excitation light E1 to E4. Here, how the fluorescence intensity of the fluorescence FL is calculated is described using an example of the fluorescence intensities ΔF1 to ΔF4 of the fluorescence FL having the polarization direction in the X direction at position A of the microchannel 220.

The fluorescence intensities ΔF1 to ΔF4 of the fluorescence FL are dependent on the pulse widths t1 to t4 of the pieces of excitation light E1 to E4, and the magnitude of the noise components due to the peripheral circuit is independent of the pulse widths t1 to t4 of the pieces of excitation light E1 to E4. Therefore, when it is defined that, with respect to two pieces of excitation light among the pieces of excitation light E1 to E4, one piece of excitation light En has a pulse width tn and a duty ratio dutyn, another piece of excitation light Em has a pulse width tm and a duty ratio dutym, the light intensity obtained from the image Pen detected by the excitation light En is An, the light intensity obtained from the image Pcm detected by the excitation light Em is Am, and the interval at which each of the pieces of excitation light E1 to E4 is emitted is T, the fluorescence intensity ΔFn of the fluorescence FL emitted from the measurement target solution by the one piece of excitation light En is represented by the Formulae (1) to (3) below where n=1, 2, 3, or 4, m=1, 2, 3, or 4, and n≠m. That is, the fluorescence intensities ΔF1 to ΔF4 of the fluorescence FL are calculated from the duty ratios dutyn and dutym of the two pieces of excitation light En and Em having different pulse widths, and the light intensities An and Am obtained from the images Pen and Pcm respectively detected by the two pieces of excitation light En and Em.

$$Δ {F}_{n} = \frac{{duty}_{n}}{\vert {duty}_{n} - {duty}_{m} \vert} \times \vert {A}_{n} - {A}_{m} \vert \qquad ( 1 )$$

$${duty}_{n} = \frac{tn}{T} \qquad ( 2 )$$

$${duty}_{m} = \frac{tm}{T} \qquad ( 3 )$$

The fluorescence intensity calculator 87 calculates the fluorescence intensity ΔFn of the fluorescence FL emitted from the measurement target solution by the one piece of excitation light En in accordance with Formulae (1) to (3). As illustrated in FIG. 8, in accordance with Formulae (1) to (3), the fluorescence intensities ΔF1 to ΔF4 of the fluorescence FL having the polarization direction in the X direction from which the noise components due to the peripheral circuit are removed from the light intensities Al to A4 (FIG. 6) obtained from the images Pc1 to Pc4 can be obtained.

In the present embodiment, the fluorescence intensities ΔF1 to ΔF4 of the fluorescence FL are calculated based on the duty ratios dutyn and dutym of the two pieces of excitation light En and Em, and the light intensities An and Am obtained from the detected images Pen and Pcm. As such, the noise components due to the peripheral circuit can easily be removed and the measurement sensitivity can be improved. Further, even if the excitation light EL includes a direct current component, the noise components due to the peripheral circuit can be removed.

Further, the excitation light intensities of the pieces of excitation light E1 to E4 are constant and the pulse widths (pulse widths t1 to t4) of the pieces of excitation light E1 to E4 modulate. As such, the excitation light EL can easily be generated and distortion of the waveform of the excitation light EL can be suppressed. With a simple configuration, the emitting (irradiation) of the excitation light EL from the light source unit 10 (light source 12) and the detection (imaging) by the CMOS image sensor 58 can be synchronized.

Note that the fluorescence intensity of the fluorescence FL having the polarization direction in the Z direction can be calculated similarly to the fluorescence intensities ΔF1 to ΔF4 of the fluorescence FL having the polarization direction in the X direction. The light intensity calculator 86 described above calculates the light intensity from one image, but the light intensity can be calculated from accumulated images by accumulating a plurality of images detected by pieces of excitation light EL having the equal pulse width.

The concentration calculator 88 of the calculator 85 calculates the degree of polarization P from the fluorescence intensity of the fluorescence FL having the polarization direction in the X direction and the fluorescence intensity of the fluorescence FL having the polarization direction in the Z direction. In the present embodiment, the fluorescence intensity of the fluorescence FL having the polarization direction in the X direction corresponds to the fluorescence intensity Ih of the fluorescence FL having the polarization direction parallel to the polarization direction of the excitation light EL. Further, the fluorescence intensity of the fluorescence FL having the polarization direction in the Z direction corresponds to the fluorescence intensity Iv of the fluorescence FL having the polarization direction perpendicular to the polarization direction of the excitation light EL. In this case, the fluorescence intensity of the fluorescence FL having the polarization direction in the X direction and the fluorescence intensity of the fluorescence FL having the polarization direction in the Z direction are the fluorescence intensity of the polarization component of the fluorescence FL emitted from the measurement target solution by the pieces of excitation light EL having the same pulse width.

The concentration calculator 88 further calculates the concentration of the detection substance from the calculated degree of polarization P and the calibration curve of the degree of polarization P and the concentration of the detection substance.

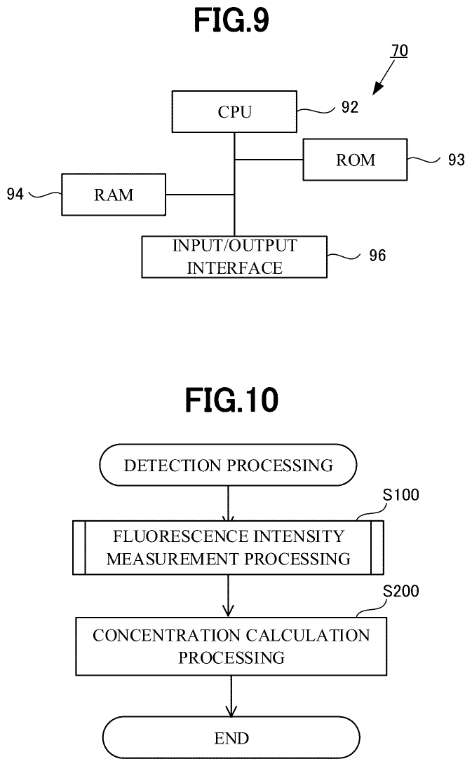

FIG. 9 illustrates the hardware configuration of the controller 70. The controller 70 includes a central processing unit (CPU) 92, a read-only memory (ROM) 93, a random access memory (RAM) 94, and an input/output interface 96. The CPU 92 executes programs stored in the ROM 93. The ROM 93 stores programs, data, and the like. The RAM 94 stores data. The input/output interface 96 inputs and outputs signals, data, and the like between the various components. The functions of the controller 70 are achieved by the execution of the programs by the CPU 92.

As described above, the fluorescence intensity of the fluorescence FL is calculated by irradiating the measurement target with the excitation light EL of which the pulse width is modulated. Therefore, the fluorescence intensity of the fluorescence FL emitted from the measurement target solution can easily be calculated by removing the noise components due to the peripheral circuit and the measurement sensitivity of the fluorescence intensity can be improved with a simple configuration. Further, since the fluorescence intensity of the fluorescence FL is calculated based on the duty ratio and the light intensity obtained from the detected image, the noise components due to the peripheral circuit can be removed even if the excitation light EL includes a direct current component.

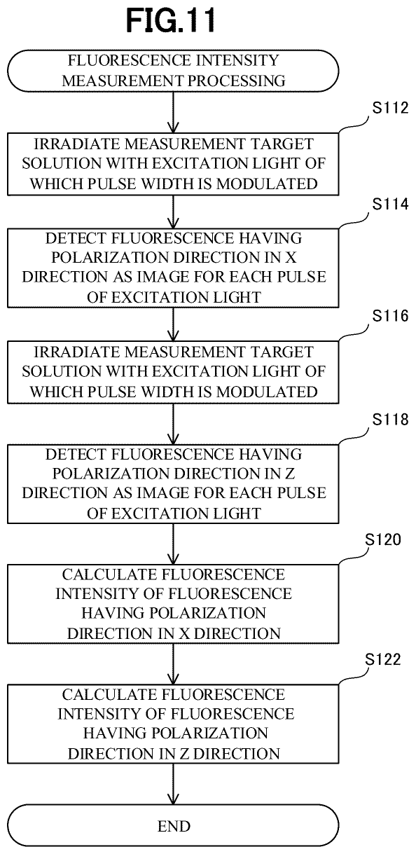

Next, detection processing of the fluorescence intensity measurement device 100 (that is, a detection substance detection method) is described with reference to FIGS. 10 and 11. As illustrated in FIG. 10, the detection processing performs, in order, fluorescence intensity measurement processing (step S100) for calculating the fluorescence intensity of the fluorescence FL, and concentration calculation processing (step S200) for detecting the concentration of the detection substance. The fluorescence intensity measurement processing (step S100) corresponds to a method of measuring a fluorescence intensity.

The fluorescence intensity measurement processing (step S100) is described with reference to FIG. 11. In the fluorescence intensity measurement processing (step S100), the fluorescence intensity of the fluorescence FL having the polarization direction in the X direction and the fluorescence intensity of the fluorescence FL having the polarization direction in the Z direction that are emitted by the measurement target solution introduced into the microchannels 220 of the microdevice 200 are calculated.

Firstly, the controller 70 controls the polarization adjustment element 54 to control the polarization direction of the fluorescence FL entered into the CMOS image sensor 58 to the X direction, and controls the light source 12 to irradiate, at the interval T, the measurement target solution introduced into the microchannels 220 of the microdevice 200 placed on the stage ST with the excitation light EL of which the pulse width is modulated and that has the polarization direction in the X direction (step S112). The light, of which the pulse width is modulated, emitted from the light source 12 is emitted, through the polarization filter 22, the excitation light filter 24, and the like, on the measurement target solution as the excitation light EL of which the pulse width is modulated and that has the polarization direction in the X direction. In the present embodiment, the pieces of pulse-shaped excitation light E1 to E4 of which the pulse widths t1 to t4 are sequentially broaden are emitted on the measurement target solution at the interval T (FIG. 5). As a result, the fluorescence FL is emitted from the measurement target solution.

Next, the controller 70 controls the CMOS image sensor 58 to detect, from among the pieces of fluorescence FL emitted from the measurement target solution, the fluorescence FL having the polarization direction in the X direction by the CMOS image sensor 58 as an image for each pulse of the excitation light EL (step S114). Specifically, the fluorescence FL emitted from the measurement target solution passes through the dichroic mirror 30, the polarization adjustment element 54, and the like, and enters the CMOS image sensor 58 as the fluorescence FL having the polarization direction in the X direction. The CMOS image sensor 58 detects, as an image, the spatial distribution of the fluorescence FL having the polarization direction in the X direction at the interval T for each pulse of the excitation light EL. The controller 70 acquires image data representing a captured image. In the present embodiment, the image Pc1 is detected by the excitation light E1 and the image Pc2 is detected by the excitation light E2. The image Pc3 is detected by the excitation light E3 and the image Pc4 is detected by the excitation light E4.

The controller 70 controls the polarization adjustment element 54 to control the polarization direction of the fluorescence FL entered into the CMOS image sensor 58 to the Z direction, and controls the light source 12 to irradiate, at the interval T, the measurement target solution introduced into the microchannels 220 of the microdevice 200 placed on the stage ST with the excitation light EL of which the pulse width is modulated and that has the polarization direction in the X direction (step S116). In this way, similarly to step S112, the measurement target solution is irradiated with the excitation light EL of which the pulse width is modulated and that has the polarization direction in the X direction, and the fluorescence FL is emitted from the measurement target solution.

Next, the controller 70 controls the CMOS image sensor 58 to detect, from among the pieces of fluorescence FL emitted from the measurement target solution, the fluorescence FL having the polarization direction in the Z direction by the CMOS image sensor 58 as an image for each pulse of the excitation light EL (step S118). Similarly to step S114, the CMOS image sensor 58 detects, as an image, the spatial distribution of the fluorescence FL having the polarization direction in the Z direction at the interval T for each pulse of the excitation light EL. The controller 70 acquires image data representing a captured image.

Next, the controller 70 calculates the fluorescence intensity ΔFn of the fluorescence FL having the polarization direction in the X direction (step S120). The controller 70 calculates the fluorescence intensity ΔFn of the fluorescence FL having the polarization direction in the X direction, from the duty ratios dutyn and dutym of the two pieces of excitation light En and Em having different pulse widths, and the light intensities An and Am obtained from the images Pcn and Pcm, of the fluorescence FL having the polarization direction in the X direction, respectively detected by the two pieces of excitation light En and Em by the Formulae (1) to (3) above where n=1, 2, 3, or 4, m=1, 2, 3, or 4, and n≠m.

Specifically, the controller 70 first calculates the light intensities Al to A4 of the microchannel 220 at the predetermined position (position A), from the images Pc1 to Pc4 from which the fluorescence intensities of the fluorescence FL having the polarization direction in the X direction are detected (FIG. 6). Next, the fluorescence intensities ΔF1 to ΔF4 of the fluorescence FL having the polarization direction in the X direction from which the noise components due to the peripheral circuit are removed from the light intensities Al to A4 are calculated from the duty ratios duty 1 to duty4 of the pieces of excitation light E1 to E4 and the calculated light intensities Al to A4 in accordance with the Formulae (1) to (3) (FIG. 8).

Next, the controller 70 calculates the fluorescence intensity of the fluorescence FL having the polarization direction in the Z direction (step S122). The controller 70 calculates the fluorescence intensity of the fluorescence FL having the polarization direction in the Z direction, from the duty ratios dutyn and dutym of the two pieces of excitation light En and Em having different pulse widths, and the light intensities obtained from the images, of the fluorescence FL having the polarization direction in the Z direction, respectively detected by the two pieces of excitation light En and Em by the Formulae (1) to (3) above where n=1, 2, 3, or 4, m=1, 2, 3, or 4, and n≠m. The specific step of calculating the fluorescence intensity of the fluorescence FL having the polarization direction in the Z direction is the same as step S120. When the processing in step S122 ends, the fluorescence intensity measurement processing (step S100) ends.

In the present embodiment, the fluorescence intensities of the fluorescence FL are calculated based on the duty ratios of the two pieces of excitation light EL, and the light intensities obtained from the images detected by the two pieces of excitation light EL. As such, the noise components due to the peripheral circuit can easily be removed and the measurement sensitivity can be improved. Further, even if the excitation light EL includes a direct current component, the noise components due to the peripheral circuit can be removed.

The excitation light EL of which the pulse width is modulated is used. As such, the excitation light EL can easily be emitted on the measurement target solution. It can also suppress the distortion of the waveform of the excitation light EL. Further, the emitting of the excitation light EL from the light source 12 and the detection by the CMOS image sensor 58 can easily be synchronized.

Returning back to FIG. 10, the concentration calculation processing (step S200) is described. The concentration calculation processing (step S200) calculates the concentration of the detection substance. First, the controller 70 calculates the degree of polarization P using the fluorescence intensity of the fluorescence FL having the polarization direction in the X direction as the fluorescence intensity Ih and the fluorescence intensity of the fluorescence FL having the polarization direction in the Z direction as the fluorescence intensity Iv. The controller 70 further calculates the concentration of the detection substance from the degree of polarization P and the calibration curve of the degree of polarization P and the concentration of the detection substance. When the concentration calculation processing (step S200) ends, the detection processing ends.

As described above, the fluorescence intensity measurement device 100 calculates the fluorescence intensity of the fluorescence FL based on the duty ratios of the two pieces of excitation light EL, and the light intensities obtained from the images detected by the two pieces of excitation light EL. As such, the noise components due to the peripheral circuit can easily be removed and the measurement sensitivity can be improved. Further, even if the excitation light EL includes a direct current component, the noise components due to the peripheral circuit can be removed.

Further, in the fluorescence intensity measurement device 100, the excitation light intensities of the excitation light EL are constant and the pulse widths of the pieces of excitation light EL modulate. As such, the excitation light EL can easily be generated. It can also suppress the distortion of the waveform of the excitation light EL. With a simple configuration, the emitting of the excitation light EL from the light source 12 and the detection by the CMOS image sensor 58 can be synchronized.

### Embodiment 2

In Embodiment 1, the pieces of pulse-shaped excitation light E1 to E4 of which the pulse widths t1 to t4 are sequentially broaden are emitted on the measurement target solution as the excitation light EL. Any method may be used to modulate the pulse width of the excitation light EL. For example, the pulse width of the excitation light EL may be randomly modulated.

The configuration of the fluorescence intensity measurement device 100 of the present embodiment is the same as the configuration of the fluorescence intensity measurement device 100 of Embodiment 1, except that the excitation light EL of which the pulse width is randomly modulated is emitted on the measurement target solution. In this embodiment, the light source controller 80, the detection controller 82, the light intensity calculator 86, and the fluorescence intensity calculator 87 of the controller 70, and the light source 12 are described.

The light source 12 of the present embodiment emits light including the excitation light EL of which the pulse width is modulated based on the PWM signal from the light source controller 80. The excitation light EL of the present embodiment has a pulse width that is randomly modulated. In the present embodiment, similarly to Embodiment 1, the excitation light EL of which the pulse width is modulated and that has the polarization direction in the X direction, is emitted on the measurement region R of the microdevice 200 through the dichroic mirror 30 and the objective lens 40. The excitation light EL of which the pulse width is modulated is described later.

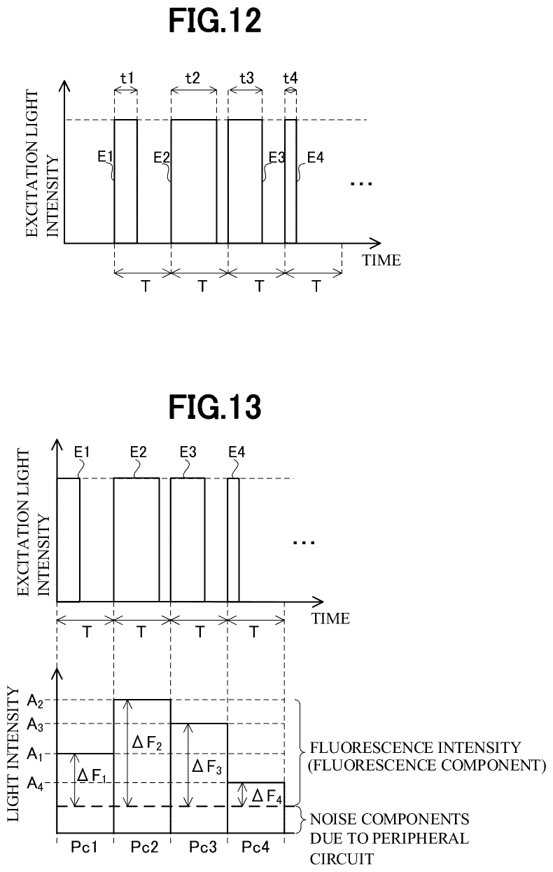

Similarly to the light source controller 80 of Embodiment 1, the light source controller 80 of the present embodiment controls the light source 12. The light source 12 of the present embodiment emits light including the excitation light EL of which the pulse width is randomly modulated based on the PWM signal from the light source controller 80. In the present embodiment, the detection controller 82, the light intensity calculator 86, and the fluorescence intensity calculator 87 are described using an example of four pieces of pulse-shaped excitation light E1 to E4 sequentially emitted, illustrated in FIG. 12.

The pieces of excitation light E1 to E4 illustrated in FIG. 12 respectively have pulse widths t1 to t4. The pulse width broadens in order of the pulse width t4 of the excitation light E4, the pulse width t1 of the excitation light E1, the pulse width t3 of the excitation light E3, and the pulse width t2 of the excitation light E2 (t4<t1<t3<t2).

Similarly to the detection controller 82 of Embodiment 1, the detection controller 82 of the present embodiment controls the CMOS image sensor 58. The CMOS image sensor 58 detects, at the interval T as an image, pieces of the fluorescence FL emitted from the measurement target solution from the pieces of excitation light E1 to E4 and having the predetermined polarization direction. In the present embodiment as well, an image detected by the excitation light E1 is defined as Pc1, an image detected by the excitation light E2 is defined as Pc2, an image detected by the excitation light E3 is defined as Pc3, and an image detected by the excitation light E4 is defined as Pc4.

Similarly to the light intensity calculator 86 of Embodiment 1, the light intensity calculator 86 of the present embodiment calculates light intensity of the microchannels 220 at the predetermined position for each of the images Pc1 to Pc4. For example, at position A of the microchannel 220 illustrated in FIG. 2, the light intensities Al to A4 illustrated in FIG. 13 are calculated.

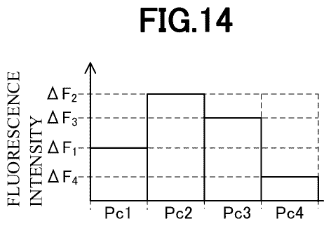

Similarly to the fluorescence intensity calculator 87 of Embodiment 1, the fluorescence intensity calculator 87 of the present embodiment calculates the fluorescence intensities ΔF1 to ΔF4 of the fluorescence FL from Formulae (1) to (3) above (FIG. 14). As described above, the fluorescence intensity of the fluorescence FL is calculated based on the duty ratios of the two pieces of excitation light EL, and the light intensities obtained from the images detected by the two pieces of excitation light EL. As such, regardless of the method of modulating the pulse width of the excitation light EL, the fluorescence intensity of the fluorescence FL can be calculated by removing the noise components due to the peripheral circuit. The excitation light EL of the present embodiment has a pulse width that is randomly modulated. As such, the influence of device-specific repeated noise can be suppressed.

In the present embodiment as well, the fluorescence intensity of the fluorescence FL is calculated based on the duty ratios of the two pieces of excitation light EL, and the light intensities obtained from the images detected by the two pieces of excitation light EL. As such, the noise components due to the peripheral circuit can easily be removed and the measurement sensitivity can be improved. Further, even if the excitation light EL includes a direct current component, the noise components due to the peripheral circuit can be removed.

In the fluorescence intensity measurement device 100 of the present embodiment, the excitation light intensities of the excitation light EL are constant and the pulse widths of the pieces of excitation light EL modulate. As such, the excitation light EL can easily be generated. It can also suppress the distortion of the waveform of the excitation light EL. With a simple configuration, the emitting of the excitation light EL from the light source 12 and the detection by the CMOS image sensor 58 can be synchronized.

### Modifications

Although the embodiments have been described above, various modifications can be made without departing from the scope of the present disclosure.

For example, in the embodiments described above, the microdevice 200 includes three microchannels 220. It is sufficient that the microdevice 200 includes at least one microchannel 220. A configuration is possible in which the microdevice 200 includes a plurality of microchannels 220.

The embodiments describe, as the excitation light EL, four pieces of pulse-shaped excitation light E1 to E4 as an example. It is sufficient that the excitation light EL is formed from pieces of pulse-shaped excitation light EL.

The excitation light EL that is linearly polarized in the embodiments is emitted on the measurement target solution (measurement target). The excitation light EL emitted on the measurement target solution may be in an unpolarized state.

The fluorescence intensity measurement device of the present disclosure may measure the fluorescence intensity of the fluorescence FL in the unpolarized state. For example, the fluorescence intensity measurement device of the present disclosure may measure the fluorescence intensity of fluorescence emitted from deoxyribonucleic acid (DNA) array. The foregoing describes some example embodiments for explanatory purposes.

Although the foregoing discussion has presented specific embodiments, persons skilled in the art will recognize that changes may be made in form and detail without departing from the broader spirit and scope of the invention. Accordingly, the specification and drawings are to be regarded in an illustrative rather than a restrictive sense. This detailed description, therefore, is not to be taken in a limiting sense, and the scope of the invention is defined only by the included claims, along with the full range of equivalents to which such claims are entitled.

What is claimed is:

1. A fluorescence intensity measurement device, comprising:
a light source to emit, to a measurement target, a plurality of pieces of pulse-shaped excitation light of which a pulse width is modulated;
a CMOS image sensor to detect, as an image for each pulse of a piece of the plurality of pieces of pulse-shaped excitation light, fluorescence emitted from the measurement target by the piece of the plurality of pieces of pulse-shaped excitation light; and
a controller to calculate a fluorescence intensity of the fluorescence, wherein
the controller calculates the fluorescence intensity of the fluorescence, based on two duty ratios of two pieces of pulse-shaped excitation light having different pulse widths of the plurality of pieces of pulse-shaped excitation light, and two light intensities obtained from the images detected by the two pieces of pulse-shaped excitation light.

2. The fluorescence intensity measurement device according to claim 1, wherein
the pulse width of the pulse-shaped excitation light emitted from the light source is randomly modulated.

3. The fluorescence intensity measurement device according to claim 1, wherein
a polarization adjustment element to adjust the fluorescence incident on the CMOS image sensor as linearly-polarized light and switch a polarization direction of the fluorescence that is linearly polarized, wherein
the CMOS image sensor detects the fluorescence having the polarization direction in a predetermined direction as the image for each pulse of a piece of the plurality of pieces of pulse-shaped excitation light, and
the controller calculates a fluorescence intensity of the fluorescence having the polarization direction in the predetermined direction.

4. A method of measuring a fluorescence intensity, comprising:
emitting, to a measurement target, a plurality of pieces of pulse-shaped excitation light of which a pulse width is modulated;
detecting, by the CMOS image sensor as an image for each pulse of a piece of the plurality of pieces of pulse-shaped excitation light, fluorescence emitted from the measurement target by the piece of the plurality of pieces of pulse-shaped excitation light; and
calculating a fluorescence intensity of the fluorescence, wherein
in the calculating,
the fluorescence intensity of the fluorescence is calculated based on two duty ratios of two pieces of pulse-shaped excitation light having different pulse widths of the plurality of pieces of pulse-shaped excitation light, and two light intensities obtained from the images detected by the two pieces of pulse-shaped excitation light.
